(19) United States
(12) Patent Application Publication
(10) Pub. No.: US 2020/0188147 A1
(43) Pub. Date: Jun. 18, 2020

(54) GEOMETRICALLY TUNABLE HYDROGEL-BASED CHEMICALLY-ELUTING SHUNT PROSTHESIS

(71) Applicant: Drexel University, Philadelphia, PA (US)

(72) Inventors: Amy L. Throckmorton, Cherry Hill, NJ (US); Kara L. Spiller, Philadelphia, PA (US); Samantha Cassel, Chalfont, PA (US); Krianthan Govender, Cupertino, CA (US); Steven G. Chopski, Philadelphia, PA (US); Randy Stevens, Philadelphia, PA (US)

(21) Appl. No.: 16/714,712

(22) Filed: Dec. 14, 2019

Related U.S. Application Data
(60) Provisional application No. 62/779,548, filed on Dec. 14, 2018.

(51) Int. Cl. A61F 2/90; A61M 27/00; A61L 31/14; A61L 31/16; C08J 3/24; A61F 2/04; A61L 31/04

(52) U.S. Cl. CPC A61F 2/90; A61M 27/002; A61L 31/145; A61M 2025/0057; C08J 3/243; A61F 2/04; A61L 31/047; A61L 31/16

(57) ABSTRACT

A shunt prosthesis comprises a synthetic tube having an inner wall defining a fixed inner diameter of the synthetic tube and a layer of hydrogel of a predetermined thickness coating the inner wall of the synthetic tube such that the layer of hydrogel has a fixed outer diameter and such that an inner diameter of the layer of hydrogel defines a diameter of a lumen extending through and defined by the shunt prosthesis. The layer of hydrogel being configured such that the predetermined thickness of the layer of hydrogel is reducable in vivo over a predetmined period of time by controlling the crosslinking density of the layer of hydrogel. A method of controlling flow through a shunt prosthesis is also provided.

### CROSS REFERENCE TO RELATED APPLICATIONS

This application claims the benefit under 35 USC § 119(e) of U.S. Provisional Patent Application No. 62/779,548, filed Dec. 14, 2018.

### BACKGROUND

In the U.S., approximately 1 million babies are born each year with a heart defect in need of corrective treatment. Of this cohort, 40,000 live births each year with congenital heart disease (CHD) require surgical intervention. In severe cases, babies are born with multiple malformations of their heart chambers and vasculature such that they are categorized to have a single ventricle (SV) physiology, such as tricuspid atresia or hypoplastic left heart syndrome (HLHS). The incidence of children born with a SV heart is about 2-4 per 10,000 births.

The treatment of SV anomalies is a formidable challenge for clinicians caring for the patients with CHD. In contrast to a normal cardiovascular physiology with two pumping chambers or ventricles, an SV physiology consists of only one functional ventricle to drive and draw blood through both the systemic and pulmonary circulations. In patients with such an anatomy, oxygenated and deoxygenated blood mix in the SV. Without surgical intervention, this set of lesions is lethal.

Hypoplastic left heart syndrome (HLHS) represents a spectrum of congenital cardiac malformations in which the left ventricle of a patient is underdeveloped and unable to support systemic circulation. HLHS affects 1 out of every 5,000 live births and has an infant mortality rate of 15-20%. After birth, the right ventricle of newborns with HLHS can provide systemic circulation via right-to-left flow through the patent ductus arteriosus. However, the duct closes a few days after birth, and, without intervention, the patient will suffer systemic circulation failure. Additionally, the pulmonary venous and systemic venous returns are mixed in the right ventricle due to the pulmonary venous return crossing the atrial septum via the patent foramen ovale. This leads to decreased oxygen saturation and cyanosis of the infant commonly referred to as blue baby syndrome.

In order to increase the survival rate, one of the main options available for HLHS patients is a three step palliative surgery to redirect blood flow which includes the Norwood, Glenn, and Fontan procedures.

Thus, as a treatment approach, the concept of a total right ventricular bypass, first introduced by Fontan and Baudet in 1968, is a palliative surgical procedure aimed at separating the systemic and pulmonary circulations, thus eliminating venous blood mixing. The current procedure creates the total cavopulmonary connection (TCPC). To compensate for the underdeveloped pulmonary circulation (i.e. high pulmonary vascular resistance and low lung volume) the TCPC is implemented in 3 stages (see FIGS. 1A, 1B and 1C), progressively separating the systemic and pulmonary circulations and gradually increasing blood flow to the lungs.

In the first stage (see FIG. 1A), a shunt or vascular prosthesis 10 is established between the systemic and pulmonary arteries (PAs), which does not prevent blood mixing but increases lung perfusion; this first surgical stage is called the Norwood procedure. The Norwood Procedure is performed within two weeks after birth and aims to repurpose the right ventricle to power systemic blood flow, while redistributing blood flow between the systemic and pulmonary circulation using shunts, such as modified Blalock-Taussig (mBT) shunts. This allows the heart to function as a single ventricle system supported by the right ventricle and involves inserting a shunt between the systemic and pulmonary circulations. Prior research has shown that the success of the Norwood Procedure hinges on proportional systemic and pulmonary flow, which is primarily dependent on shunt geometry.

A modified Blalock-Taussig (mBT) shunt allows for blood flow from the innominate artery to the right pulmonary artery via a small synthetic tube. The shunt is left in the infant for 4-6 months until the patient has grown a sufficient amount to undergo the Glenn procedure, when it is removed. Typically, Blalock-Taussig shunts have a 3.5 mm inner diameter. For the average infant, the 3.5 mm inner diameter allows for maximum oxygen delivery to tissues due to adequate flow distribution between systemic and pulmonary circulation immediately after implantation. However, this does not continue to be provided as the child grows and the oxygen requirements and flow parameters change.

The shunt currently used in the Norwood procedure for HLHS patients is the GORE PROPATEN Vascular Graft, which is made of expanded polytetrafluoroethylene (ePTFE). It is designed to address thrombosis in small diameter grafts by using the Carmeda BioActive Heparin Surface, which covalently bonds heparin to the ePTFE luminal surface, which leaves the bioactive heparin intact. The graft has been shown to have improved thromboresistance compared to ePTFE grafts without heparization. While this graft addresses thrombosis, the graft has a fixed diameter, so it does not account for flow changes in a developing infant.

These patients require a delicate balance of blood flow to each of the now parallel systemic and pulmonary circulations. Serious complications, like hypoxia, frequently result from instabilities in the blood oxygenation, from either overperfusion or hypoperfusion of the pulmonary arteries as delivered through the shunt. A too-narrow shunt diameter has an increased internal resistance, which may lead to pulmonary hypoperfusion, higher shear stresses, and irregular blood flow patterns that boost the risk of hemolysis and thrombosis. On the other hand, a shunt that is too large in diameter has a lower internal resistance, which draws more blood volume to the lungs and away from the systemic circulation, adversely impacting the oxygenation of systemic end organs. Thus, it is critically important to select an appropriate shunt size for these patients.

Conventionally, commercially available fixed diameter-shunts are utilized by surgeons in the Norwood procedure. The shunt type, diameter, length, and orientation are determined during surgery, and all dictate the flow balance between the lungs and body. However, the decisions made during surgery are complicated by the expected rampant growth and development that the child experiences during this initial phase of life. These changes, such as vessel growth, cardiac output, and pulmonary vascular resistance, also alter the blood oxygenation balance as the patient develops.

Clinicians manage the balance of blood oxygenation post-Norwood using pharmacological therapies, catheterization, or surgical corrections. While pharmacological management is routine, physical intervention (including dilation, stents, or surgical correction) has been reported necessary in 28% of cases due to shunt complications. The complex nature of this delicate oxygenation balance is further illustrated by the mortality rate associated with the Norwood procedure, which varies in reports between 7-19%. Compared to other surgeries for heart defects, the mortality rates for the Norwood are among the highest, and have not improved in recent years despite advancements in the field and in overall patient care.

The high risk of complications and the lack of dramatic improvements highlights a fundamental flaw in the use of this shunt; a fixed diameter shunt cannot maintain adequate blood oxygenation during the numerous physiological changes that occur in the first few months of life. A shunt geometry that balances blood oxygenation during developmental growth is needed.

The above referenced shunt or vascular prosthesis 10 is removed in the second stage (see FIG. 1B—the Glenn Procedure), and the superior vena cava (SVC) is connected to PAs, resulting in a bidirectional cavopulmonary connection (BCPC) and partial right ventricular bypass. The TCPC is completed in the third stage (see FIG. 1C—the Fontan Procedure) with the inferior vena cava (IVC) connected to the BCPC.

The Fontan completion produces a configuration in which a SV drives and draws blood through the systemic circulation and then further through the pulmonary vascular beds. The SV experiences a lower preload pressure and a subsequent increase in venous pressure to compensate for the lack of a pressure boost typically provided by a right ventricle. This altered physiologic state is known as the “Fontan paradox.” It has been shown that late morbidity is related to these higher systemic venous pressures, especially in the inferior systemic veins. The higher the vascular resistance downstream of the liver, the higher the pressure difference required to achieve a given cardiac output. Thus, the absence of this right ventricle in Fontan patients places significant limitations on the amount of energy available to drive blood through the pulmonary vascular beds.

Modifications to the Fontan procedure, coupled with better clinical management, have improved surgical outcomes reducing post-operative mortality to the level of simpler CHD repairs. However, patients are subjected to long-term complications, such as thrombosis, atrial arrhythmias, ventricular dysfunction, and protein losing enteropathy. Over their lifespan, these high-risk patients utilize healthcare resources disproportionate to their numbers. Collectively, their hospitalization costs exceed $1.4 billion/year, constituting an emerging public health concern. Those with SVs represent a large percentage of those adults with CHD and are a problematic subgroup. Moreover, the number of adults with severe CHD rivals the pediatric population.

In addition to those patients with SVs, many other congenital or acquired cardiovascular anomalies, diseases, or related diagnoses warrant the use of a blood-contacting shunt configuration in their treatment strategy.

### SUMMARY

According to an aspect of the present invention, a shunt prosthesis or similar implant device comprises a synthetic tube or like substrate. The synthethic tube has an inner wall defining a fixed inner diameter. A layer of hydrogel of a predetermined thickness coats the inner wall of the synthetic tube such that the layer of hydrogel is affixed to the inner wall of the synthetic tube and such that the thickness of the layer of hydrogel defines a diameter of a lumen extending through and defined by the shunt prosthesis. The thickness of the layer of hydrogel is reducable in vivo over a predetmined period of time in a controlable manner by control of a crosslinking density of the layer of hydrogel. Additionally, a thin film of stretchable and easily deformable Teflon, polyurethane, or comparable material may line the hydrogel layer on the outer diameter surface, the inner diameter surface, or both surfaces for any axial length from the inlet of the shunt to the outlet of the shunt. In this manner, an autonomously expanding shunt is provided, such as for use with pediatric patients.

As an alternative for geometric adjustment, light exposure may be used as a photo-activation source to activate cross-linking and to retract the inner lumen of the shunt, thereby increasing the inner diameter in a general and local spatial effect. This may be accomplished by cardiac catheterization.

According to another aspect of the present invention, a method of controlling flow through a shunt prosthesis is provided. The method comprises the step of enlarging a diameter of a lumen of the shunt prosthesis implanted within a patient in vivo. The shunt prosthesis comprises a synthetic tube or like substrate having an inner wall defining a fixed inner diameter of the synthetic tube and a layer of hydrogel of a predetermined thickness coating the inner wall of the synthetic tube such that a thickness of the layer of hydrogel defines a diameter of the lumen. The enlarging step of the method is accomplished by reducing the thickness of the layer of hydrogel in vivo over a predetmined period of time by activating or altering a crosslinking density in the layer of hydrogel.

### BRIEF DESCRIPTION OF THE DRAWINGS

Various features of the embodiments described in the following detailed description can be more fully appreciated when considered with reference to the accompanying figures, wherein the same numbers refer to the same elements.

FIGS. 1A-1C are views of the three stages of surgical repair of functional single ventricle physiology of a heart.

FIGS. 2A-2J are views showing examples of common vascular shunt configurations for the State I Norwood surgical repair of functional single ventricle physiology of a heart.

FIG. 3A is a cross-sectional view of a shunt prosthesis having a layer of hydrogel according to an embodiment.

FIG. 3B is an enlarged section of the shunt prosthesis of FIG. 3A showing the release or activation of a crosslinking agent within the layer of hydrogel via the temporal degradation or activation of microstructures according to an embodiment.

FIG. 3C is a cross-sectional view of the shunt prosthesis of FIG. 3A after the crosslinking density of a hydrogel layer is altered or activated which thereby increases the diameter of the lumen defined by the shunt prosthesis according to an embodiment.

FIG. 4A is a cross-sectional view of a shunt prosthesis having a layer of hydrogel according to an embodiment.

FIG. 4B is a cross-sectional view of the shunt prosthesis of FIG. 4A after the crosslinking density of a hydrogel layer is altered or activated which thereby increases the diameter of the lumen defined by the shunt prosthesis according to an embodiment.

FIG. 5 is a perspective partially cut-away view of a shunt prosthesis having a layer of hydrogel according to an embodiment.

FIGS. 6A-6C are cross sectional views of an alternate embodiment of a shunt prosthesis having a plurality of concentric layers of hydrogel separated by dissolvable intermediate hydrophobic polymer layers.

FIGS. 7A-7C are cross sectional views of an alternate embodiment of shunt prosthesis having a concentric layers of hydrogel separated by peel away intermediate hydrophobic polymer layers according to an embodiment.

FIG. 8 is a graph showing a Power-Law Model of HLHS patient growth versus shunt flow rate.

FIG. 9 is a schematic view showing cross-sections of a shunt according to an embodiment before and after crosslinker release.

FIG. 10 is an image of an experimental prototype of hydrogel adhered to a fixed rigid other surface.

FIG. 11 is a graph showing scaled percent change of inner diameter of the hydrogel versus time upon varying glutaraldehyde concentration.

FIG. 12 is a graph showing scaled percent change of inner diameter of the hydrogel versus time upon varying gelatin weight percent.

### DETAILED DESCRIPTION

For simplicity and illustrative purposes, principles of embodiments are described herein by referring primarily to examples thereof. In the following description, numerous specific details are set forth to provide a thorough understanding of the embodiments. It will be apparent to one of ordinary skill in the art that the embodiments may be practiced without limitation to these specific details.

“Patient” or “subject” as used herein means a mammalian animal, including a human, a veterinary or farm animal, a domestic animal or pet, and animals normally used for clinical research. More specifically, the subject of these methods, systems and devices is a human.

Embodiments disclosed herein are intended for all patients that require the placement of a blood-contacting or biofluid-contacting shunt, patch, or the like in the cardiovascular or other systems. The embodiments generally relate to an implantable device, such as a shunt prosthesis for a biofluid, such as blood.

As an example, according to one contemplated embodiment, a blood-contacting shunt prosthesis for use in the cardiovascular system of a patient is provided such that a diameter of a lumen defined by an inner wall of the shunt prosthesis is designed to gradually enlarge or widen over a period of time (i.e., to provide a lumen of greater cross-sectional area) to permit greater flow, such as blood flow, therethrough. For example, in the case of a newborn baby having a heart defect, the inner lumen of an implanted shunt may be designed to gradually widen over a period of time to permit increased blood flow as the newborn grows and his/her circulatory volume increases.

By way of example, an autonomously expanding shunt for a pediatric patient may be provided. This may include a modified Blalock Taussig (mBT) shunt with a diameter that expands uniformly over time, allowing for increased pulmonary perfusion needed due to patient growth. Such a shunt will provide appropriate flow and pressure profiles for Norwood patients without generating excessive shear stress or residence times. Thus, a novel geometrically tunable blood shunt may be provided for recipients of the Norwood procedure, a palliation for single ventricle malformations. The shunt design addresses limitations of the current treatment by adjusting in proportion to the patient's growth, allowing for greater control over the balance of blood flow through the shunt in response to various physiological changes.

Despite advancements, mortality rates for the Norwood procedure remain relatively high. The use of a fixed shunt during infancy is inherently insufficient, due to the physiological changes that alter the fluid conditions through the shunt. In contrast, the tunable design of embodiments disclosed herein offers a new methodology to rebalance blood flow in these delicate patients.

According to an embodiment, a geometrically tunable hydrogel-based blood shunt is provided. The shunt has a geometrically expandable inner diameter that can be modified in proportion to the growth and development of the patient, thus offering a new approach to intrinsically adjust the ratio of blood oxygenation. The design of this device includes a fixed outer sheath coated internally with a thick layer of a hydrogel. Hydrogels are 3D crosslinked polymer networks that are swollen with water. They are known for their biocompatibility and anti-fouling properties, making them ideal choices for blood-contacting biomaterials such as catheters and shunts. In addition, their degree of swelling can be easily controlled by manipulating their crosslinking, in that decreasing the degree of crosslinking causes increased swelling. By increasing the degree of crosslinking in a hydrogel-coated inner lumen of a shunt, swelling is expected to decrease, resulting in an overall increase in lumen diameter. Therefore, blood flow can be modulated by controlling hydrogel crosslinking. Hydrogel crosslinking may be controlled through the infusion of crosslinking agents or the controlled release of crosslinking agents from within the hydrogel itself.

According to a contemplated embodiment, the inner wall of a shunt prosthesis or other substrate is coated with at least one layer of a hydrogel such that the layer of hydrogel has a fixed outer diameter where it adheres to a surrounding substrate. The geometry of the hydrogel layer (i.e., the inner diameter, length, thickness, etc.) may be caused to be altered by chemically-induced crosslinking as a result of a crosslinking agent reacting with the hydrogel material at a controlled rate.

For instance, the crosslinking agent may be carried in a microstructure embedded within the hydrogel layer and gradually released therefrom over time for reaction with the hydrogel. The gradual release of the crosslinking agent over time causes the thickness of the hydrogel layer to contract (i.e., causes the inner diameter to move toward the fixed outer diameter of the hydrogel layer). Accordingly, the embodiment enables temporal control of the geometry of the shunt prosthesis (namely, the diameter or radius of the lumen defined within the shunt prosthesis by the hydrogel layer) by the control of crosslinking density of the hydrogel.

Thus, a shunt may include a hydrogel material that is bound to an outer rigid layer. Drug delivery microspheres or the like may be embedded within the hydrogel that and contain a crosslinker solution which will be released over time to crosslink the hydrogel, causing the inner diameter to expand.

Another option for geometric adjustment is light exposure as a photo-activation source to activate cross-linking and to retract the inner lumen of the shunt, thereby increasing the inner diameter in a general and local spatial effect. This could be accomplished by cardiac catheterization.

By way of example, shunts according to embodiments disclosed herein may be designed such that an inner diameter of an annular cross-section thereof may autonomously expand radially in diameter by about, for example, 15-18% over time to account for patient growth over time. This autonomous, transient inner diameter increase may allow for increase blood flow to the pulmonary system. However, the average outer diameter change over time of the shunt may be limited to being lower than about 1%. In addition, the inner material of the shunt cross section must remain adhered to the rigid outer layer, since detachment would skew inner diameter expansion results.

The shunt must also be able to provide blood flow to the pulmonary system without creating high levels of blood damage, and therefore needs to maintain a blood damage index (BDI) less than 0.2%. The blood damage index is used to quantify the level of damage that erythrocytes experience, and can be calculated based on scalar shear stress and residence time using a power law developed from regression analysis of experimental data]. The above referenced flow profile requirements help ensure proper flow distribution through the shunt and reduces the possibility of thrombosis or hemolysis in the patient.

Contemplated targeted patient populations for such a device include children with single ventricle physiology and all patients that require the placement of a shunt in the cardiovascular system. For reasons discussed above, mortality after the first-stage Norwood operation for hypoplastic left heart syndrome is high. The underlying etiology of this unstable SV physiology can be attributed directly to the systemic arterial shunt. Decompensation to heart failure is typically of sudden onset and is unpredictable, which underscores the vulnerability of these infants. Sudden death ceases to be an obstacle in the second and third stage operations, and outcomes are much improved.

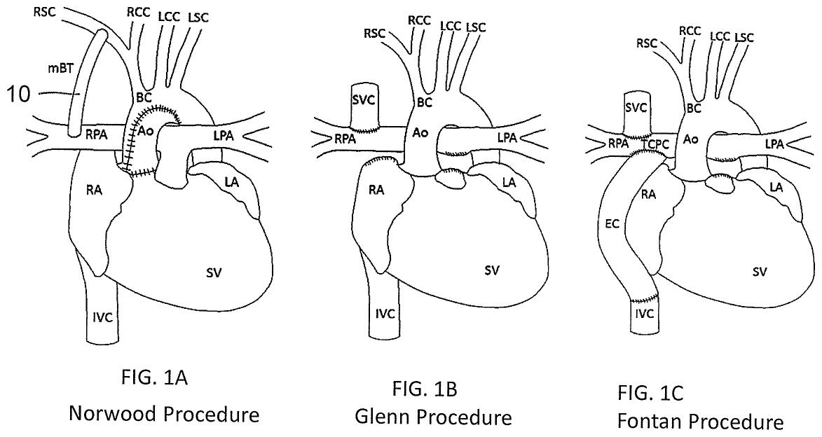

In the stage-1 Norwood operation (see FIG. 1A), the aortic arch is enlarged, and the ductus arteriosus is divided. The PAs are separated from the heart above the pulmonary valve. The reconstructed aorta is then attached to the pulmonary valve to provide unobstructed flow from the morphologic right ventricle to the systemic circulation. The native aortic root is joined as a side-branch to provide flow to the coronary arteries. Blood ejected from the ventricle is a mixture of oxygenated (left atrial blood, which crosses an atrial septal defect to enter the right atrium) and deoxygenated blood (from the vena cavae). Blood flow to the PAs, which are disconnected from the heart, is derived from the high-pressure systemic arterial circulation using a systemic-to-pulmonary arterial shunt 10 (see FIG. 1A).

The systemic-to-pulmonary arterial shunt, at the Norwood stage, creates multiple physiological challenges for the underdeveloped and compromised SV which must: (1) adequately support both pulmonary and systemic circulations in an unstable parallel configuration; (2) eject and deliver twice the normal ventricular volume to feed both the pulmonary and systemic circulations; (3) perform this excessive workload under severely hypoxemic conditions (PaO₂ 30-40 mmHg); and (4) achieve all of these under conditions of impaired myocardial perfusion due to a decreased diastolic blood pressure from shunt run-off and increased myocardial wall tension due to volume overload.

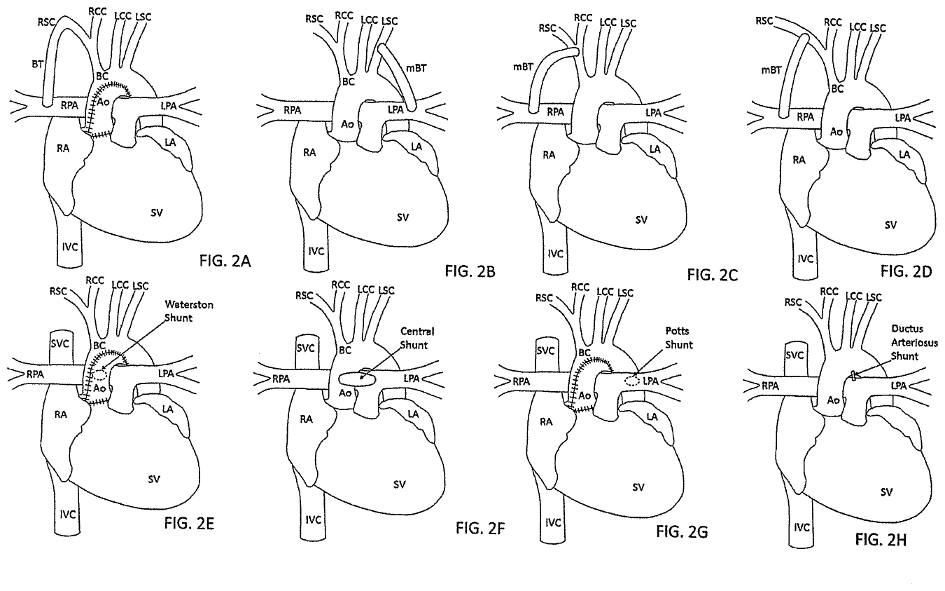

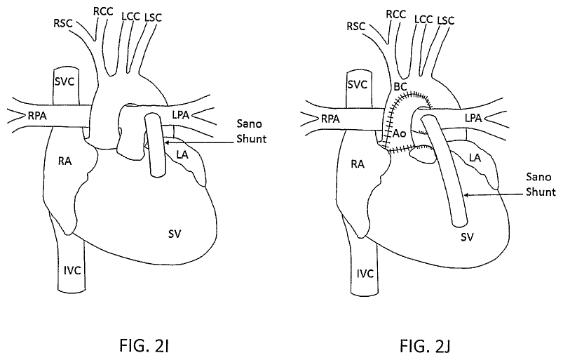

As examples, FIGS. 2A-2J illustrate common systemic arterial shunts or prosthetic connections during the Norwood procedure in the palliative treatment of SV physiology. Although these conventional shunts are the current treatment strategies, mortality at this stage is high, and there are significant limitations associated with their selection and usage during the Norwood stage. New and innovative therapeutic solutions are needed not only for patients with CHD, but also those having shunts that are placed in contact with any biofluids in the human body.

Conventional commercial and synthetic shunts have inherent challenges achieving the above referenced four required clinical treatment objectives and limitations in their clinical usability. There is innate subjectivity in selecting the length and diameter that is most appropriate for the given patient and his/her anatomic heterogeneity in anticipation of a development trajectory. The shunt must have a low risk of thrombosis and enable growth and development of the infant.

The presence of the constant-diameter shunt may exacerbate the deleterious conditions that mandate its use, which are hypoxic pulmonary vasoconstriction and pulmonary hypertension. It may impair pulmonary vascular maturation and may detrimentally elevate pulmonary vascular resistance. Perturbations that impact the balance of blood flow in the parallel circulations require compensation elsewhere in the circulation to restore equilibrium. Instability can arise suddenly, such as when hypoxemia leads to lung hypoperfusion, and thus worsening hypoxemia. Moreover, higher PaO₂ results in the vasodilation of the pulmonary circulation, leading to overperfusion and additional elevation of PaO₂. Management of these Norwood patients is challenging and sometimes requires contradictory treatment strategies, including the reduction of inspired oxygen and hypoventilation. These confounding limiting factors of constant-diameter shunt usage contribute the mortality levels and instability of Norwood patients.

The Norwood procedure is very likely to be performed on infants who are born with single ventricle physiology in the near-term (typically with 24-48 hours and less than 1 week) after birth. Conventionally, a commercially available, constant-diameter shunt or vascular prothesis is selected and surgically placed connecting the innominate artery and the right pulmonary artery or the brachiocephalic artery to the undivided right pulmonary artery in order to increase blood flow to the lungs and to increase oxygen transport to the peripheral tissues. This constant-diameter shunt prosthesis may remain inside of the patient from infancy until early infancy. During this time period, the patient undergoes significant growth and development in this time span. Reoperation may occur to either replace or remove the shunt and, in many cases, to perform further surgical repairs or reconstruction to the cardiovascular anatomy.

According to embodiments disclosed herein, a geometrically-tunable, hydrogel-based, chemically-eluting vascular prosthesis is provided to address the limitations of conventional static shunts. The shunt prothesis according to embodiments is biocompatible, adjusts in geometric shape in proportion to the growth and development of an infant, has non-thrombogenic properties, incorporates hydrogel-based chemistry, can better balance a distribution of volume between pulmonary and systemic circulations, and can serve as a new therapeutic option for thousands of babies with severe CHD. In addition, the shunt can be used and placed in contact with any human biofluids. The shunt solves significant problems that are incurred with the conventional static, fixed-diameter and fixed-length shunts that are made of Teflon or other materials.

A shunt prosthesis according to embodiments may have one or more of the following features.

Physiologic fluid flow—the shunt prosthesis according to embodiments may be designed to support fluid flow in all physiologic applications, including lymphatic circulatory, cerebral spinal, urine production and elimination, ocular, salivary, and blood flow conditions.

Rapid deployment—the shunt prosthesis according to embodiments may be designed to be deployed through percutaneous insertion techniques including in a cardiac catheterization lab, by laparoscopic surgical or minimally invasive surgical approaches, during the conventional Norwood procedure in patients with a SV physiology or category of congenital heart defects that warrant use of a blood flow shunt, and other related physiologic fluid flow applications.

Novel hydrogel-based and chemically-eluting construct—the inner wall of the shunt according to embodiments may be covered by a layer of hydrogel matrix which is temporally chemically modified.

A hydrogel structure is a 3-D array of polymer chains swollen with water. The greater the chain length between crosslinks, the larger the mesh size of the hydrogel, and the more swollen the structure can become. The process of crosslinking hydrogels decreases the mesh size and therefore decreases the hydrogel's ability to swell. This decrease in swelling potential causes the hydrogel to shrink.

According to embodiments, the hydrogel-based shunt may have unique polymer microstructures embedded within its walls. The geometry (diameter, length, thickness, etc.) of the shunt changes due to chemically induced crosslinking as a result of a crosslinking agent being released from the embedded microstructures and then reacting with the hydrogel material. The crosslinking agent may be released from within the hydrogel at a controlled rate. This agent could be entrapped in microstructures that are strategically dispersed and immobilized within the hydrogel. The density of these microstructures may vary depending upon the targeted desired mechanical properties of the shunt, and thus the desired geometry.

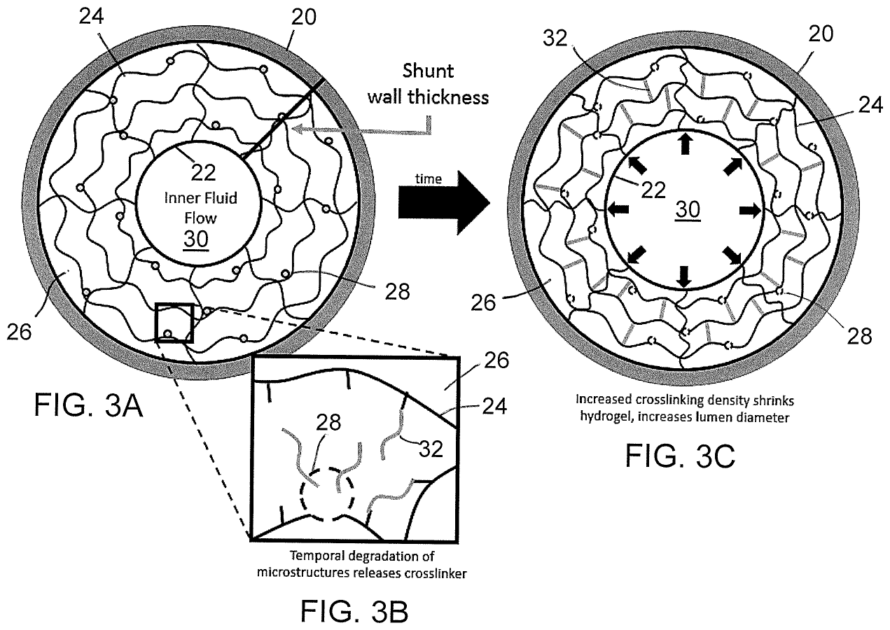

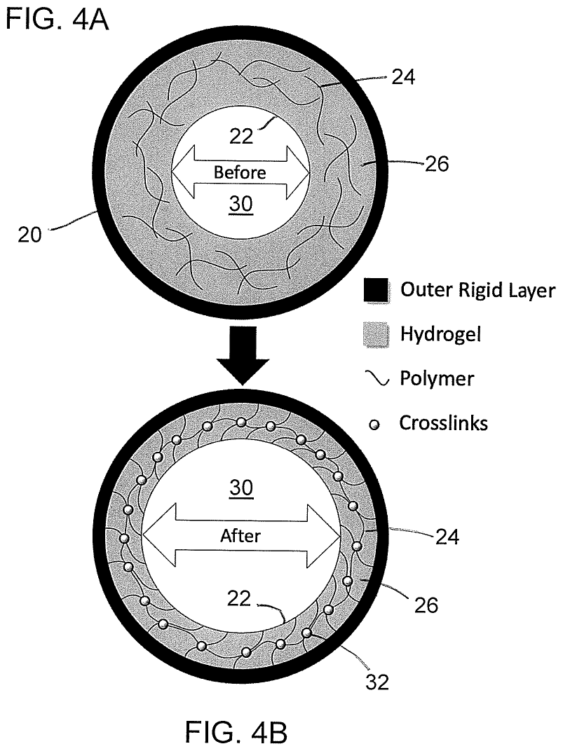

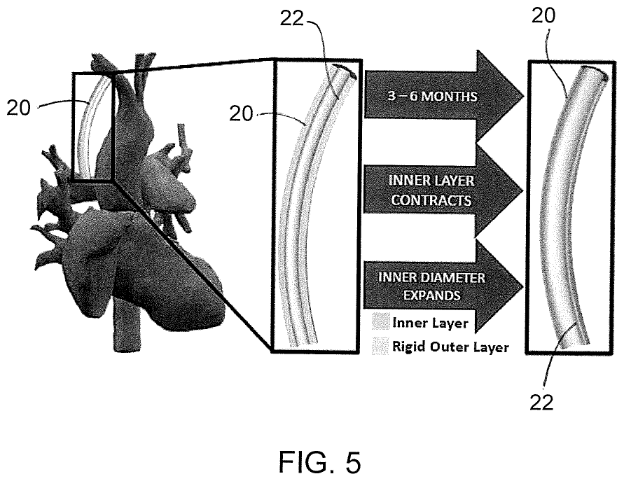

FIG. 3A shows a cross-sectional view of the shunt 20 of an embodiment before inner diameter 22 increase. FIG. 3A shows the polymer lattice 24 of the hydrogel 26, microstructures 28 represented as circles, and the inner lumen 30 for fluid flow. Gradual degradation or activation of the microstructures 28 over a period of time releases the crosslinker 32 at a controlled rate, for instance, as best shown in FIG. 3B. The crosslinker 32 covalently reacts with the hydrogel polymer 26, creating new connections within the lattice 24, decreasing the mesh size thereof (see FIG. 3C). This increase of crosslink density leads to a more compact hydrogel structure, increasing shunt inner lumen diameter 22, as shown in FIG. 3C. FIGS. 4A and 4B show similar views of the shunt 20 before and after crosslinking, respectively. FIG. 5 shows the shunt 20 as connected to a heart, an enlarged cut-away view showing the size of the inner diameter 22 of the shunt 20 before crosslinking, and an enlarged cut-away view showing the size of the inner diameter 22 of the shunt 20 after crosslinking.

Alternate embodiments of methods of implementation of the above concept may include any of the following.

Control over hydrogel crosslinking density—the increased lumen diameter may be accomplished by designing the hydrogel to experience an increase in crosslinking density over time, thus causing de-swelling in a physiological environment. As an example, annuli comprised of gelatin-based hydrogels, were affixed at their outer edges to non-swellable substrates and immersed in solutions of increasing concentrations of glutaraldehyde, a crosslinking agent for gelatin hydrogels. Hydrogel crosslinking density increased in an amount proportional to glutaraldehyde concentration, resulting in proportional increases in the inner lumen diameter.

Temporal control over increasing crosslinking density can be accomplished intrinsically by embedding degradable nano- or micro-structures within the hydrogel matrix that slowly release crosslinking agent at a rate dependent on degradation of the nano- or micro-structures as discussed above. For example, PLGA nanoparticles incorporating glutaraldehyde may be embedded within gelatin hydrogel, so that release of glutaraldehyde occurs over about 4 to 6 weeks in vivo as the PLGA hydrolytically degrades. Alternatively, degradation may occur through other biocompatible means, such as enzymatically.

Temporal control over increasing crosslinking density can be accomplished extrinsically through intravenous administration to the patient of crosslinking agent at user-defined time points. As an example, a clinician may use imaging modalities or prior experience to determine an optimal time point at which an increased shunt lumen diameter would be desirable. At this point the clinician may inject crosslinking agent to react with the hydrogel material without affecting other cells or tissues in the body. This injection could be delivered intravenously or within the shunt using minimally-invasive means of access. This strategy may be used with or without a light source to facilitate hydrogel crosslinking. Alternatively, a clinician may use laparoscopic, intravascular, intravenous minimally-invasive means of access and surgical tools to peel away layers of material to increase the inner diameter of the shunt.

Regardless of the method of introducing the crosslinking agent (intrinsic vs. extrinsic), it is critical that the crosslinking agent is bio-orthogonal, meaning that it cannot adversely react with any naturally occurring chemical groups in the body, so that there is no risk of unintentional crosslinking outside of the hydrogel material.

Another option for geometric adjustment is light exposure as a photo-activation source to activate cross-linking and to retract the inner lumen of the shunt, thereby increasing the inner diameter in a general and local spatial effect. This may be accomplished by or during cardiac catheterization.

Hydrogel crosslinking chemistries—the shunt should be compatible with any hydrogel crosslinking chemistry. For example, poly(ethylene glycol) (PEG)-based hydrogels may be prepared with pendant vinyl functionalities; intrinsic or extrinsic administration of bifunctional linkers with thiol groups may crosslink with the vinyl groups through thiol-ene reactions. Another example is the use of a hydrogel polymer functionalized with tetrazine derivatives so that addition of bis-trans-cyclooctene causes crosslinking. Other useful reactions include strain-promoted alkyne azide cylcoaddition and tetrazine-norborene.

Hydrogel crosslinking density (and thus tunable geometric changes) may be controlled by varying the polymer molecular weight or concentration, the number of reactive functionalities, the crosslinking agent concentration, polymer degradability, hydrogel osmotic pressure, and the rate of addition of crosslinking agent (whether intrinsic or extrinsic). The hydrogel-lined shunt according to embodiments should have the ability for temporal control over their geometry and should enable control over increasing crosslinking density over time.

As a further alternative, the shunt could be provided such that it enables layer-by-layer degradation to increase a lumen for fluid flow. Thus, another method to achieve increasing lumen diameter over time is to provide a shunt with linings forming layers of surface-eroding biodegradable polymers, so that degradation of each layer results in removal of the polymer and increased lumen diameter. An exemplary polymer that could be used in this case is poly(glycerol sebacate) (PGS). However, many surface-eroding polymers are highly hydrophobic (thus thrombogenic) and/or degrade into byproducts that would accumulate in sufficient volumes for the intended application that they would be problematic for the patient. Thus, this strategy may be used in combination with a hydrogel strategy to reduce the total amount of polymer required.

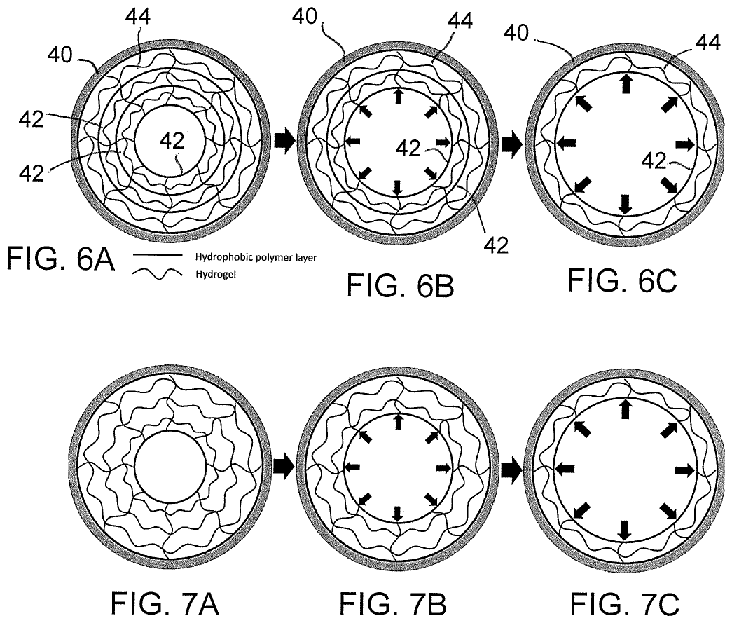

In the above referenced alternative, a shunt 40 has biodegradable hydrogel layers 44 separated by thin layers of hydrophobic polymers 42 that prevent degradation of the hydrogel layers 44 until the hydrophobic polymer 42 degrades (see FIGS. 6A-6C). The potential for thrombogenecity of the hydrophobic polymer separators 42 is reduced by incorporating zwitterionic functional groups or other modifications that increase wetting. In this case, layers 42 could be dissolved or even peeled away as described before. The clinician may use laparoscopic, intravascular, intravenous minimally-invasive means of access and surgical tools to peel away layers of material to increase the inner diameter of the shunt (for instance, see FIGS. 7A-7C).

A shunt prosthesis according to embodiments, may also have one or more of the following features.

Tunable geometrically shape alteration—the shunt prosthesis, given its chemical construct, may be able to dynamically and temporally adjust its geometric shape (i.e. length, diameter, wall thickness, chemical composition, etc.), for any of these goals: (a) in proportion to the growth, development, and flow needs of the patient; (b) maintenance of improved balance of fluid flow distribution; (c) redirection of physiologic fluid flow in accordance with desired treatment strategies; and (d) preservation of the system's driving pressure profile of the fluid.

Biocompatibility—the fluid contacting surfaces of the prosthesis should not adversely impact and damage cells in the physiologic fluid or tissue in direct contact with the shunt.

Non-thrombogenic and non-inflammatory properties—the fluid contacting surfaces of the prosthesis should not activate platelets, damage blood cells, or stimulate complement or coagulation factors such that thromboemboli are generated. There should be no adverse inflammatory response due to the presence of the shunt in contact with any biofluid.

Time dependent properties—the distinctive and vast combination of chemical construct for the shunt prosthesis should create a versatile shunt configuration that may provide changing mechanical properties. This may be due to the controlled release of the crosslinking agent in the microstructures and the placement and density of the microstructures in the shunt wall.

### EXAMPLE

### Mathematical Modeling and Reasoning

### Hagen-Poiseuille Flow

For general fluid flow, the mathematical model typically used is the Navier-Stokes equations. These equations relate the momentum, energy transfer, and mass transfer of a fluid system. However, for purposes herein, the flow through a modified Blalock-Taussig shunt can be modeled as Hagen-Poiseuille flow. Hagen-Poiseuille flow is typically used for laminar flow through a pipe, a geometry which is analogous to the mBT shunt discussed above. The three main assumptions for this model are that there are no body forces, the fluid has constant density, and laminar flow is purely in the axial direction.

The parabolic velocity profile through the shunt can be evaluated by applying the no-slip boundary condition at the static shunt walls and recognizing the axis of symmetry at the center of the shunt. The velocity of the fluid is related to the volumetric flow rate through the cross-sectional area of each layer of fluid, which ultimately can be expressed in accordance to Equation (1).

$$Q = \frac{{d}_{0}^{4} π Δ P}{128 µL} \\ {d}_{0} = pipe diameter \\ μ = dynamic viscosity \\ Δ P = pressure drop across pipe \\ L = axial length \qquad ( 1 )$$

This Hagen-Poiseuille flow model equation shows a fourth power dependency between the volumetric flow rate and shunt diameter, making it the most sensitive parameter in this flow-pressure relationship and therefore plays an important role in the success of the shunt within the patient.

### Blood Damage

The introduction of a theoretical shunt needs to be compatible with the altered circulatory system. To quantify the excessive damage to erythrocytes passing through the proposed mBT shunt, the blood damage index (BDI) is used to measure of the change in hemoglobin relative to the total hemoglobin content in blood. The model used to calculate the blood damage index is derived from data gathered from a Couette viscometer experiment conducted by Heuser et al. (Heuser G, Opitz R. “A Couette viscometer for short time shearing of blood.” Biorheology, vol. 17, pp. 17-24, 1980). The data gathered in Heuser's paper was fit to a power-law mathematical model, which is displayed in Equation (2) below:

$$\frac{dHb}{Hb} = 1.8 \times {10}^{- 6} \cdot {τ}^{1.991} \cdot {T}^{0765} \\ \frac{dHb}{Hb} = BDI \\ τ = scalar stress \\ T = residence time \qquad ( 2 )$$

This model incorporates both the residence time and scalar stress, the latter of which is calculated from the stress tensor in accordance with Equation (3) below:

$$τ = {[ \frac{1}{6} \sum ( {σ}_{ii} - {σ}_{jj} ) ( {σ}_{ii} - {σ}_{jj} ) + \sum {σ}_{ij} {σ}_{ij} ]}^{\frac{1}{2}} \\ {σ}_{ii} = {σ}_{jj} = principle stresses \\ {σ}_{ij} = shear stresses \qquad ( 3 )$$

### Infant Growth

In order to gauge the mBT shunt radial change needed for an aging patient, a model must be developed to characterize the appropriate flow rates needed over time. However, no mathematical model to predict HLHS patient growth currently exists, and therefore, a growth model was developed. The first step is identifying the most critical parameter used to describe flow conditions for HLHS patients, which is the flow ratio, defined in Equation (4). Based on literature, it was determined that the optimal flow ratio for Norwood procedure patients is between 0.9 and 1.

$$Flow Rate = \frac{{Q}_{P}}{{Q}_{S}} \\ {Q}_{P} = pulmonary blood flow \\ {Q}_{S} = systemic blood flow \qquad ( 4 )$$

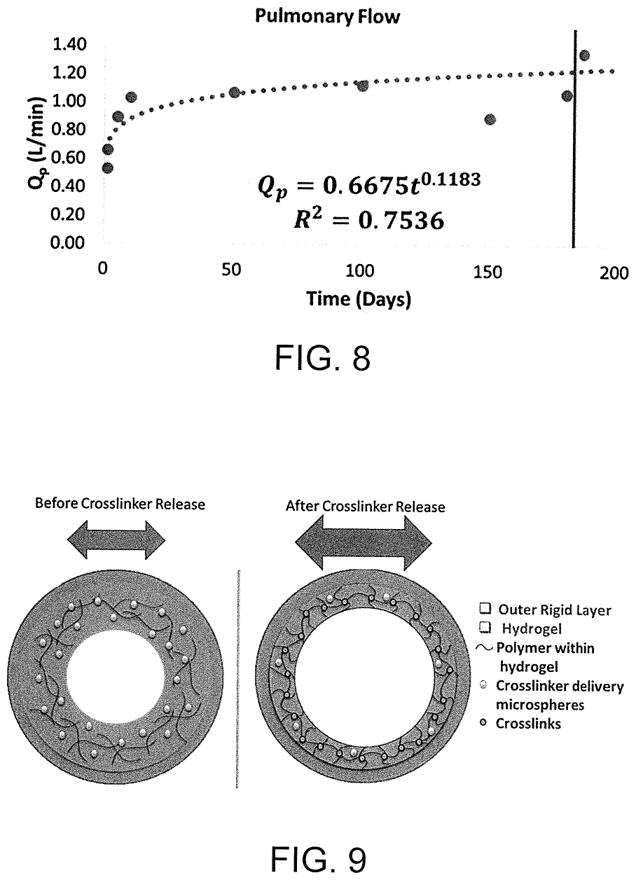

Subsequently, a growth curve, with a flow ratio of 0.9, was derived from data gathered from published sources. The equation for the power-law developed is displayed in FIG. 8, with the vertical line representing the point at which the patient reaches six months of age.

### Overview

The proposed solution consists of a rigid outer shell that will sustain a fixed diameter and an inner region that will consist of a hydrogel material (see FIG. 9). A hydrogel is a network of hydrophilic polymers that swells with water. A chemical crosslinker solution is administered to the hydrogel by a crosslinker delivery system that will cause the hydrogel material to contract. Chemical crosslinking is a process of intermolecular bonding a polymer and a chemical through covalent bonds. As the number of crosslinks increases in the hydrogel, more bonds will form between the polymer and the crosslinker and water will be expelled from the hydrogel, causing the hydrogel to contract, and the inner diameter to expand. As the inner diameter expands, the outer layer will remain rigid allowing only expansion of the inner diameter and not the outer diameter.

The ability of this shunt to expand over time is important. Over a six-month span, an infant will grow significantly and there are currently no shunts that expand to account for flow changes in a growing infant. This design will slowly expand over the six months that the shunt will be implanted, which will decrease complications currently seen with HLHS patients and decrease mortality.

### Specifications

The hydrogel material that is used in this example is gelatin, which is a natural polymer that results from the degradation of collagen. Gelatin is inexpensive and easily forms a hydrogel upon physical and chemical crosslinking, making it an ideal polymer to use to study the effects of a chemical crosslinker on the diameter of an annular hydrogel.

Glutaraldehyde is a common crosslinker that is used in a variety of tissue engineering applications. Glutaraldehyde reacts easily with gelatin to form covalent crosslinks at room temperature. Glutaraldehyde crosslinks polymers by forming covalent bonds with the amine groups on the polymer. The aldehyde group of the glutaraldehyde reacts with the amino group of the gelatin to form a Schiff base linkage (═CHN═). The outer casing of the hydrogel will be made of poly(methyl methacrylate) (PMMA) sheets. The PMMA sheets will need to be chemically modified to give it amine functional groups. The PMMA sheets will then be able to interact with the amine groups of the gelatin through the development of covalent bonds. This covalent bond attaching the gelatin hydrogel to the PMMA sheet will allow a fixed outer diameter to be maintained during crosslinking.

To allow for autonomous expansion of the shunt, poly(lactic-co-glycolic acid) (PLGA) microspheres will encapsulate the glutaraldehyde crosslinker solution and will be embedded within the gelatin hydrogel. As the PLGA microspheres degrade via hydrolysis, the glutaraldehyde will be released from the microspheres and will diffuse into the gelatin hydrogel. At this point, the glutaraldehyde will be able to crosslink the gelatin polymer and allow for inner diameter expansion.

### Prototype

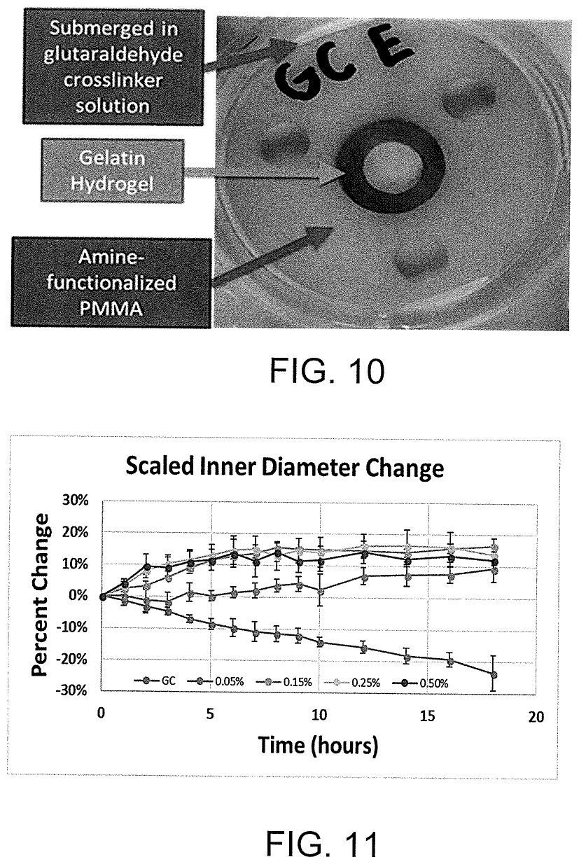

In order to construct the prototype, a four-plate mold was conceptualized and cut out of PMMA using a laser cutter. The center two plates contained a central hole for a PTFE dowel, while the top plate was the yellow outer rigid material for each sample. After the chemical surface modification of the top plate, M5 screws and nuts were used to connect and compress the four plates together. Gelatin solution was then pipette into the resulting cavity and allowed to gelate, before the mold was deconstructed. FIG. 10 shows an example of the prototype used for verifying inner diameter change, without incorporation of PLGA microspheres.

### Design Verification

### Experimental Verification Procedure

The goal of the experimental verification is to verify that an inner diameter change of 15-18% can be controlled using gelatin and glutaraldehyde. The deliverable for the experimental verification will be an annular hydrogel with the outer diameter fixed to a rigid outer surface and a dynamic inner diameter that represents the cross section of an mBT shunt (see FIG. 10).

The first two experiments were performed using the same procedure to determine the ideal gelatin weight percent and glutaraldehyde crosslinker concentration to achieve the required expansion. To perform these experiments, the rigid PMMA outer surface underwent chemical surface modification through aminolysis. This was accomplished by first submerging all PMMA top sheets into a 1 M ethylenediamine-DMSO solution in a beaker for 20 minutes. Next, gelatin solutions were prepared in PBS and allowed to gelate. These hydrogels were pre-swollen in PBS to reach equilibrium for 48 hours. The samples were then placed in wells and submerged in varying concentrations of glutaraldehyde crosslinker in PBS. Images of the hydrogels were taken over time to track inner diameter changes. These images were filtered using a 2D high pass Gaussian filter and analyzed in MATLAB to quantify the change. From this, the relationship between crosslinker concentration, gelatin weight percent and inner diameter change was determined to optimize the inner diameter expansion between 15-18%. This novel method was utilized, because there is no current engineering standard for determining hydrogel inner diameter changes. The method will be validated by quantifying error of the MATLAB code/image capture and experimental data using statistics.

### Surface Area Scaling

The dimensions that were used in the prototype do not reflect the current mBT shunt sizes used by today's surgeons. In order to compare the inner diameter change of the prototype to the expected inner diameter change of an implantable shunt, a surface area relationship had to be developed. Since the amount of hydrogel crosslinking depends directly on the number of functional groups, the volume change between the prototype and a theoretical shunt cross section will be the same, if the gel composition does not change. Since the height of the samples would not change between the prototype and the theoretical cross section, this also means that the surface area change will be the same. Based on the same surface area change, the results of the experimental prototypes can be scaled based on the relationship in Equation (5) below:

$$Δ {d}_{t} = \frac{\sqrt{( 2 Δ {d}_{p} + Δ {d}_{p}^{2} ) {d}_{i , s , p}^{2} + {d}_{i , s , t}^{2}}}{{d}_{i , s , t}} - 1 \\ Δ {d}_{p} = change in prototype diameter \\ Δ {d}_{t} = change in theoretical diameter \\ {d}_{i , s , p} = starting inner diameter of prototype \\ {d}_{i , s , t} = starting inner diameter of theoretical shunt \qquad ( 5 )$$

### Experimental Verification Results

The first experiment was to assess the effect of varying glutaraldehyde crosslinker concentration solutions on the inner diameter expansion. The glutaraldehyde concentrations used for this test were 0%, 0.05%, 0.15%, 0.25%, and 0.50%. The samples were prepared using a 10% w/v gelatin solution in PBS. There was also a surface modification control in this experiment that did not undergo the aminolysis step of the protocol.

These diameter changes were compared to the initial time point and displayed as percent change over time (see FIG. 11). For all concentrations of glutaraldehyde, the gelatin hydrogel showed an increase in inner diameter, with higher concentrations showing a larger increase due to an increase in crosslinks (see FIG. 11). The glutaraldehyde control showed a decrease in inner diameter (see FIG. 11), showing that the non-crosslinked hydrogel swelled in the presence of PBS. The surface modification control also showed to have very varied data, indicating that the surface modification was necessary to adhere the gelatin to the PMMA. This validates the concept of increasing the inner diameter of a hydrogel by introducing a crosslinking agent. However, the 10% gelatin hydrogels did not achieve the required 15-18% inner diameter change.

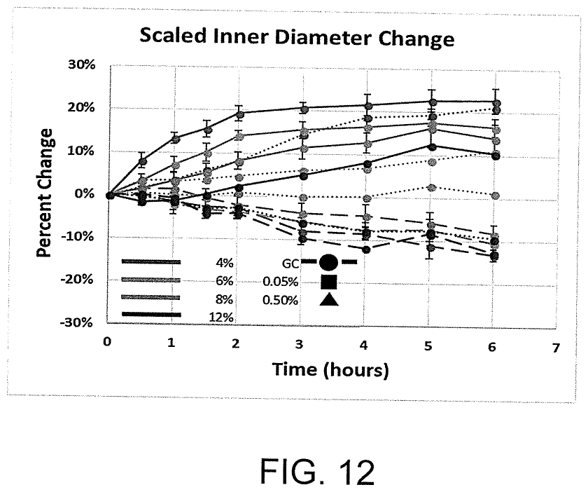

The second experiment was to determine the effect of gelatin weight percent on the inner diameter expansion. The samples were prepared using 4, 6, 8, and 12% w/v gelatin solutions in PBS. The inner diameter change from this experiment is shown in FIG. 12. The largest inner diameter changes occurred at 0.5% glutaraldehyde and 4% w/v gelatin with a maximum of 20% change observed (see FIG. 12) after scaling the data using Equation 5. The 4 and 6% gelatin hydrogels achieved the required 15-18% change with the 0.50% glutaraldehyde crosslinker solution, while the 8 and 12% gelatin hydrogels did not. Additionally, all glutaraldehyde controls (GC) swelled over time in the absence of crosslinker indicating a successful positive control (see FIG. 12). In addition, it was observed that the higher the glutaraldehyde concentration and the lower the gelatin weight percent the larger the diameter change. Lastly, there was no significant percent change of the gelatin outer diameters for all gel conditions.

### Autonomous System Verification Procedure and Results

To address autonomous constraint, a verification test was conducted to evaluate the feasibility of a drug delivery system for the delivery of the crosslinker to the hydrogel polymer network. PLGA microspheres were fabricated using a water/oil/water double emulsion method. There were two conditions of microspheres. One condition was prepared with 25% glutaraldehyde and another was a control which contained PBS. Microspheres were suspended in PBS and were gently shaken at 37° C. for 2 weeks. Glutaraldehyde release from microspheres and activity was evaluated by applying supernatant from the suspension after microspheres were pelleted to a gelatin sample. Colorimetric analysis of these gelatin samples indicated that there was no quantifiable release of glutaraldehyde from the microspheres over the course of two weeks.

### Computational Verification

In addition to experimental verification, tests using ANSYS CFX software were performed. Computational verification methods are utilized to develop flow-pressure relationships, and to calculate blood damage indices. The overall process consists of creating a fluid domain using SolidWorks, which is then imported and meshed in ANSYS Workbench and subsequently put through the solver.

### Discussion

The experimental verification results indicated that the required inner diameter expansion of 15-18% was achieved by externally applying a glutaraldehyde crosslinker solution of 0.5% (v/v) with 4% or 6% wt gelatin solutions. Due to the superior mechanical integrity and ease of fabrication of the 6% gelatin compared to the 4% gelatin observed during the fabrication process, it was determined that a 6% gelatin would be the best specification for the design of the specifications tested.

When the feasibility of the PLGA glutaraldehyde crosslinker delivery system was investigated, the results indicated that glutaraldehyde release was unsuccessful. This could be due to a variety of reasons including having a low encapsulation efficiency of glutaraldehyde, glutaraldehyde interactions with PLGA leading to inefficient degradation of PLGA or inactive glutaraldehyde, or a combination of these reasons.

The computational verification results indicated that the BDI of streamline particles flowing through the shunt was less than the limit of 0.2%. These results indicate that the shunt does not cause hemolysis, so red blood cells passing through the shunt domain do not experience overly adverse stresses. This is important because erythrocyte damage in these already unstable pediatric patients can cause complications.

The shunt according to embodiments disclosed herein should provide new therapeutic options for thousands of babies with severe CHD who undergo multiple open-heart staged surgical procedures, as well as new therapeutic options for all patients who require a shunt placement in contact with biofluids.

While the principles of the invention have been described above in connection with specific devices, systems, and/or methods, it is to be understood that this description is made only by way of example and not as limitation. One of ordinary skill in the art will appreciate that various modifications and changes can be made without departing from the scope of the claims below.

For example, the above described invention may be provided as any conduit or patch that grows (involutes) with the patient over time. In addition, the invention may be used for neo artery formation such that an outside of a device is provided as a long-term permeable ePTFE skeleton with the inside being a hydrogel seeded with autologous cells. The hydrogel may be used as a layering to grafts, such as for artificial valves and the like. The shunt prosthesis may be used as a neural tube as a possible wrap to place near the area of injury and allow time to be dissolved and replaced with repair tissue. The components of the hydrogel may favor or inhibit the cellular repair. A layer of hydrogel may be 3-D printed and may provide a form of artificial skin. In this manner, it may be placed on skin cells as a protective layer or as a solid layer with micro-perforations to provide a host for new skin growth. The prosthesis may be used as single ventricles. The hydrogel may be used as a coating on heart valves, a coating on VAD tubing, a coating on pacer wires to shrink and allow for removal, a coating inside the wires that allows more wires in the future (inside the clear layer), or as a building block for any chemical.

Still further, the outside of the prosthesis may be PTFE, the inside may be provided with a hydrogel, and the hydrogel may be mixed with a drug that prevents atherosclerosis or inflammation or sends signals to increase angiogenesis or prevents clot. The hydrogel could also be used to decrease shear stress by allowing endothelial cells to coat the entire graft not just fibroblasts or platelets or the like. The prosthesis could also be utilized as a drug eluting stent having a hydrogel coating with a drug, such as paclitaxel. The stent could be designed as described above to increase in radius over time to provide increased coronary blood flow. Thus, as the paclitaxel disappears, the larger diameter or radius increases flow and decreases the chance of future thrombus.

The specification and figures are to be regarded in an illustrative rather than a restrictive sense, and all such modifications are intended to be included within the scope of the present invention.

1. A shunt prosthesis, comprising a synthetic tube having an inner wall defining a fixed inner diameter of said synthetic tube and a layer of hydrogel of a thickness coating said inner wall of said synthetic tube such that said layer of hydrogel is affixed to said inner wall of said synthetic tube and such that the thickness of said layer of hydrogel defines a diameter of a lumen extending through and defined by the shunt prosthesis, said layer of hydrogel being configured such that the thickness of said layer of hydrogel is reducable in vivo over a predetmined period of time by control of crosslinking density of said layer of hydrogel.

2. The shunt prosthesis according to claim 1, wherein the layer of hydrogel has a mesh structure provided by a three-dimensional array of polymer chains and crosslinks in which the greater a length of the polymer chains between crosslinks, the larger a mesh size of the hydrogel.

3. The shunt prosthesis according to claim 2, wherein chemically-induced crosslinking provided by a reaction of the hydrogel with a crosslinking agent decreases the mesh size of the mesh structure of the layer of hydrogel and thereby reduces the thickness of the layer of hydrogel and increases the diameter of the lumen.

4. The shunt prosthesis according to claim 2, wherein photo-activation with a light source activates crosslinking of the hydrogel to decrease the mesh size of the mesh structure of the layer of hydrogel and to thereby reduce the thickness of the layer of hydrogel and increase the diameter of the lumen.

5. The shunt prosthesis according to claim 1, further comprising polymer microstructures embedded, dispersed and immobilized within the layer of hydrogel, said polymer microstructures containing a crosslinking agent.

6. The shunt prosthesis according to claim 5, wherein the polymer microstructures are configured to degrade over a predetermined period of time to gradually release the crosslinking agent within the layer of hydrogel and thereby decrease the thickness of the layer of hydrogel and gradually increase the diameter of the lumen over the predetermined period of time.

7. The shunt prosthesis according to claim 1, wherein the layer of hydrogel comprises a plurality of concentric layers of hydrogel.

8. The shunt prosthesis according to claim 7, wherein the plurality of concentric layers of hydrogel are separated by a peelable or disolvable hydrophobic polymer layer.

9. A method of controlling flow through a shunt prosthesis, comprising the step of enlarging in vivo a diameter of a lumen of a shunt prosthesis implanted within a patient, the shunt prosthesis comprising a synthetic tube having an inner wall defining a fixed inner diameter of the synthetic tube and a layer of hydrogel of a thickness coating the inner wall of the synthetic tube such that the thickness of the layer of hydrogel defines a diameter of the lumen, wherein said enlarging step is accomplished by reducing the thickness of the layer of hydrogel in vivo over a predetmined period of time by altering a crosslinking density of the layer of hydrogel.

10. The method according to claim 9, wherein the layer of hydrogel has a mesh structure provided by a three-dimensional array of polymer chains and crosslinks in which the greater a length of the polymer chains between crosslinks, the larger a mesh size of the hydrogel.

11. The method according to claim 10, wherein said enlarging step is accomplished by chemically-induced crosslinking provided by a reaction of the layer of hydrogel with a crosslinking agent to decrease the mesh size of the mesh structure of the layer of hydrogel and thereby reduce the thickness of the layer of hydrogel and increase the diameter of the lumen.

12. The method according to claim 10, wherein said enlarging step is accomplished by photo-activation with a light source to activate crosslinking of the hydrogel to decrease the mesh size of the mesh structure of the layer of hydrogel and to thereby reduce the thickness of the layer of hydrogel and increase the diameter of the lumen.

13. The method according to claim 9, wherein the layer of hydrogel has polymer microstructures embedded, dispersed and immobilized therein, said polymer microstructures containing a crosslinking agent.

14. The method according to claim 13, wherein the polymer microstructures are configured to degrade over the predetermined period of time to gradually release the crosslinking agent within the layer of hydrogel and thereby decrease the thickness of the layer of hydrogel and gradually increase the diameter of the lumen over the predetermined period of time.

15. The method according to claim 9, wherein the layer of hydrogel comprises a plurality of concentric layers of hydrogel, wherein the plurality of concentric layers of hydrogel are separated by a peelable or disolvable hydrophobic polymer layer which is peeled or disolved before said enlarging step.
